(19) United States
(12) Patent Application Publication
(10) Pub. No.: US 2004/0038429 A1
(43) Pub. Date: Feb. 26, 2004

(54) Method of searching for novel lead compound

(76) Inventors: Shuichi Hirono, Tokyo (JP); Kazuhiko Iwase, Saitama (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/416,338

(22) PCT Filed: Nov. 13, 2001
(86) PCT No.: PCT/JP01/09890

(30) Foreign Application Priority Data
Nov. 14, 2000 (JP) 2000346229

(51) Int. Cl. G06F 19/00; G01N 33/48; G01N 33/50; G01N 31/00; G01N 33/543

(52) U.S. Cl. 436/518; 702/19; 702/22

(57) ABSTRACT

The invention provides a technique for finding out compounds with potential to become leads in the compounds of medicinal drugs, agricultural chemicals, etc.

In the retrieval of compounds with potential to become leads, a novel method for finding out compounds with potential to become leads is provided by performing the superposition between conformers of compounds and extracting pharmacophores, specifying the common functional groups and their positions and searching compounds by three-dimensional data base retrieval, and subjecting hit compounds to docking with the virtual receptor model, which is evaluated with the value of interaction energy and the value of strain energy.

### TECHNICAL FIELD

In the compounds for medicinal drugs, agricultural chemicals, etc., as one of methods for retrieving compounds that can become leads, a method wherein fit compounds are retrieved from three-dimensional data base, based on particular functional groups or their positions of known compounds, which are known to bind to the same proteins such as enzyme and receptor, is known. However, going a step further, an effective method for extracting compounds with potential to become leads from compounds having hit by retrieval is needed.

### BACKGROUND TECHNOLOGIES

In the compounds for medicinal drugs, agricultural chemicals, etc., as the method for retrieving compounds that can become leads, a method wherein fit compounds are retrieved from three-dimensional data base, based on particular functional groups or their positions of known compounds, which are known to bind to the same proteins such as enzyme and receptor, is known. Further, as the methods for extracting compounds with potential to truly become leads from compounds having hit by retrieval, Chem-X (OMG, Inc.) that uses rmsd (root-mean-square deviation, nanometer) of distances between particular functional groups, ISIS-3D (MDL Information Systems, Inc.) that uses the number of rotatable bonds, rmsd of distances between particular functional groups and intramolecular van der Waals interaction energy, CATALYST (MSI, Inc.) that uses the extent of geometrical fit to pharmacophore model, etc. are known. For appropriately evaluating the hit compounds, however, it is required to properly calculate the values of van der Waals interaction energy and electrostatic interaction energy that work between receptors and compounds. RECEPS (Institute of Medicinal Molecular Design) is evaluating with the value of goodness of fit by making out a receptor space with data of compound on the lattice points around template compound and by inserting the retrieving compounds into that receptor space.

The process for extracting pharmacophores by superposing between conformers of compounds can be performed by using a novel method that superposes the molecular structure of compounds such as medicinal drugs and agricultural chemicals, described, for example, in Jap. Kokai Tokkyo Koho, JP 010,298,113. Since long ago, the pharmacophore is explained to be “The molecular framework that carries the essential features responsible for a drug's biological activity” (P. Ehrlich, Dtsch. Chem. Ges., 42,17, 1909). This model that three-dimensionally and geometrically arranged the atoms and the functional groups common to compounds that bind to enzyme and receptor is the pharmacophore model.

For the extraction of pharmacophores, in the cases of, for example, thrombin inhibitors; NAPAP, 4-TAPAP and MQPA, it is possible to simultaneously presume their binding (active) conformations and pharmacophores from round robin superpositions of respective 113, 457 and 202 conformers (K. Iwase and S. Hirono, Journal of Computer-Aided Molecular Design, 13, 499-512,1999). These superpositions were those that reproduced the superposition of inhibitor molecules in the state bound to enzyme proteins, obtained from X-ray crystallography. Moreover, virtual receptor models that use these compounds as templates can be made out by using, for example, Receptor (MSI, Inc.). Thevirtual receptormodel is a model that complementarily depicts the natures of binding compounds such as shape, partial charge, hydrophobicity and hydrogen bond on the surface of virtual receptor, different from the pharmacophore model. The search of compounds by the three-dimensional data base retrieval permits to flexibly retrieve the corporate compound data base and available chemical directory (MDL Information Systems, Inc.) by using, for example, ISIS-3D (MDL Information Systems, Inc.) based on the pharmacophores presumed from previous superposition. At this time, it is possible to roughly narrow down by cutoff of the number of rotatable bonds, rmsd and value of van der Waals energy.

The hit compounds obtained by the three-dimensional data base retrieval are inserted into the virtual receptor model constructed previously to perform initial docking. Concretely, at first, the conformations of compounds having hit by ISIS-3D are output by CFS (Conformationally Flexible Searching) -SD file. Then, superposition of hit compounds on the compounds used for the construction of virtual receptor model is performed, using the coordinate of atoms (3 atoms ordinarily) designated by the conditions of three-dimensional retrieval. In this process, it is possible to perform the initial docking of hit compounds with virtual receptor model. This manipulation itself is not a novel technique and is applicable, if a program for superposing point to point is available. For performing the optimum docking, it is required to perform the calculation for energy optimization of each hit compound under the constrained conditions of virtual receptor model. From the virtual receptor model and hit compounds in the optimum docking state determined in this way, the value of interaction energy and the value of strain energy are evaluated, making it possible to select the hit compounds. The interaction energy is the sum total of van der Waals energy and electrostatic energy between virtual receptor model and the compound, and the more negative it becomes, the stronger the interaction becomes. The strain energy is an internal strain energy of the compound when docking with virtual receptor, and the closer to zero, the more stable.

In the compounds for medicinal drugs, agricultural chemicals, etc., as one of the methods for retrieving compounds that can become leads, a method wherein fit compounds are retrieved from three-dimensional data base, based on particular functional groups or their positions of known compounds, which are known to bind to the same proteins such as enzyme and receptor, is known. However, going a step further, a method for selecting compounds with potential to become leads from compounds having hit by retrieval is needed.

### DISCLOSURE OF THE INVENTION

For solving the subject as described above, the inventors have found a novel technique wherein superposition is performed between conformers of compounds and pharmacophores are extracted, the common functional groups and their positions are specified to search compounds by three-dimensional data base retrieval, and hit compounds are allowed to subject the docking with virtual receptor model, which is evaluated with the value of interaction energy and the value of strain energy, leading to the completion of the invention.

Namely, in the retrieval of compounds with potential to become leads, the invention is a novel method for finding out compounds with potential to become leads, by

(1) sampling conformers of known compounds,

(2) performing the superposition between conformers of known compounds and making out virtual receptor model from superposed conformers of known compounds,

(3) extracting pharmacophores from superposed conformers of known compounds,

(4) making out the conditions of three-dimensional retrieval based on the extracted pharmacophores to perform three-dimensional data base retrieval,

(5) superposing the conformers of compounds having hit by three-dimensional retrieval based on the conditions of threedimensional retrieval, making superposed conformers of known compounds as targets, and converting the coordinate,

(6) inserting conformers of hit compounds with the coordinate converted into the virtual receptor model constructed in (2) described above and performing the initial docking, and

(7) determining the optimum docking from the optimization of structure of hit compounds subjected to initial docking in (6) described above under the constrained conditions of virtual receptor model and evaluating the value of interaction energy/value of strain energy between virtual receptor model and compounds.

The invention relates to a method wherein, in the compounds for medicinal drugs, agricultural chemicals, etc., fit compounds are retrieved from three-dimensional data base, based on particular functional groups or their positions of known compounds, which are known to bind to the same proteins such as enzyme and receptor, and compounds with potential to become leads are selected effectively from hit compounds.

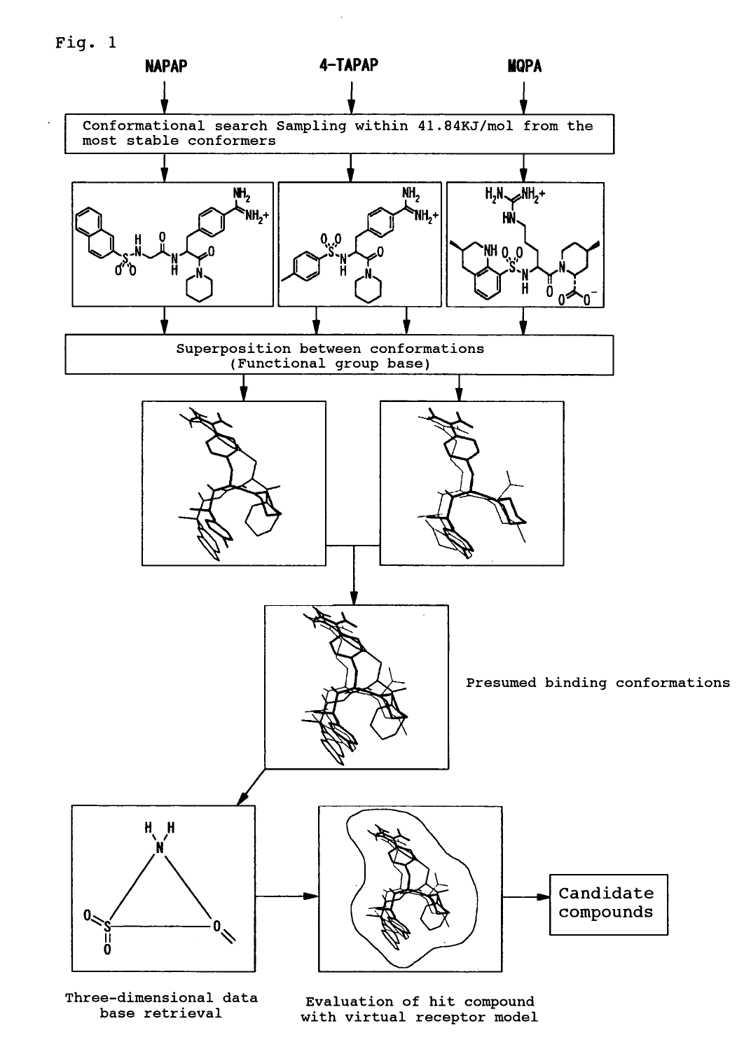

The process of the three-dimensional retrieval is described in FIG. 1. In following, each step will be explained.

The conformational search of known compounds which are known to bind to the same proteins such as enzyme and receptor is performed, and conformers within, for example, 41.84 to 62.76 KJ/mol from the most stable values of energy are sampled among the conformers generated. The conformers of respective compounds are superposed in the mode of round robin, using the superposing method described in Jpn. Kokai Tokyyo Koho JP 010,298,113 to presume their active conformations and pharmacophores. At this time, a plurality of pharmacophores are presumed, but, practically, it is required to narrow down by classifying into several groups from the value of rmsd between pharmacophores or further from the result made full use of the technique of statistical analysis. The construction of virtual receptor model becomes therefore to perform by using presumed active conformation in a certain group.

The three-dimensional retrieval of compounds is performed by making out the conditions of retrieval based on the pharmacophores and flexibly retrieving the corporate compound data base and available chemical directory (MDL Information Systems,. Inc.), using ISIS-3D (MDL Information Systems, Inc.). At the time of retrieval, it is possible to roughly narrow down by cutoff of the number of rotatable bonds, rmsd and value of van der Waals energy. Moreover, by loosely setting the condition of distance, it is also possible to perform the retrieval that simultaneously satisfies a plurality of pharmacophores.

The initial docking of hit compounds with virtual receptor model was determined by superposing between compounds used for the construction of virtual receptor model with hit compounds based on the three-dimensional coordinate of atoms (3 atoms ordinarily) designated by the conditions of three-dimensional retrieval. At this time, the conformation of compounds having hit by ISIS-3D and the three-dimensional coordinate used were those output by CFS (Conformationally Flexible Searching) -SD file. For performing the optimum docking, calculation for the energy optimization of each hit compound is performed under the constrained conditions of virtual receptor model. From the optimum docking determined in this way, hit compounds can be selected making, for example, the fact that the value of interaction energy is negative and the fact that the value of strain energy is 62.76 KJ/mol or less as criterions.

Besides, this method is also applicable to the construction of initial docking model of conformers of hit compounds by the three-dimensional retrieval with three-dimensional model of proteins.

### BEST EMBODIMENT TO PUT THE INVENTION INTO PRACTICE

Using the superposing method described in Jpn. Kokai Tokyyo Koho JP Hei10-298113, the 113, 457 and 202 conformers of thrombin inhibitors having structures shown in Table 1;

MQPA: (2R,4R)-4-methyl-1-[Nα-(3-methyl-1,2,3,4-tetrahydro-8-quinolinesulfonyl)-L-arginyl]-2-piperidinecarboxylic acid 4-TAPAP: Nα-(4-toluene-sulfonyl)-DL-p-amidinophenylalanyl-piperidine

NAPAP: Nα-(2-naphthyl-sulfonyl-glycyl)-DL-p-amidinophenyl-alanyl-piperidine were superposed, respectively, to presume the active conformations and pharmacophores of inhibitors.

TABLE 1

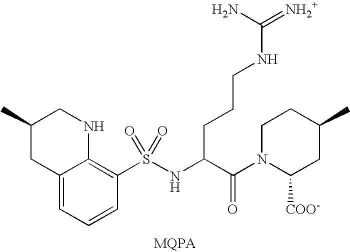

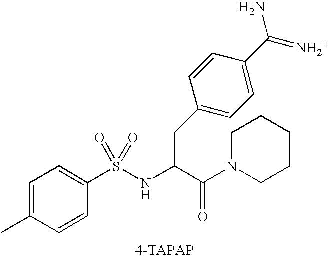

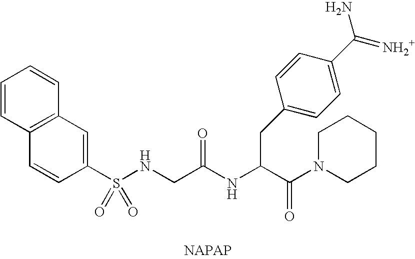

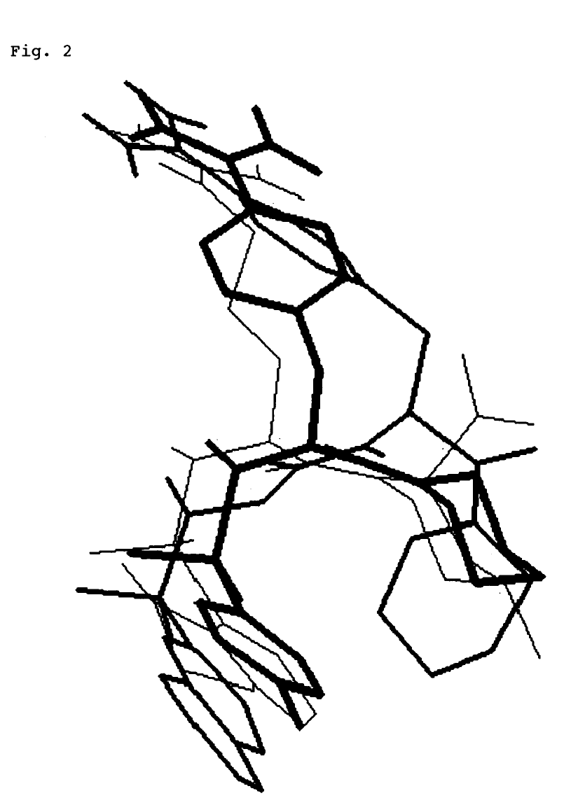

Utilizing one set (FIG. 2) among some sets of presumed active conformations, the construction of virtual receptor model was performed using Receptor (MSI, Inc.).

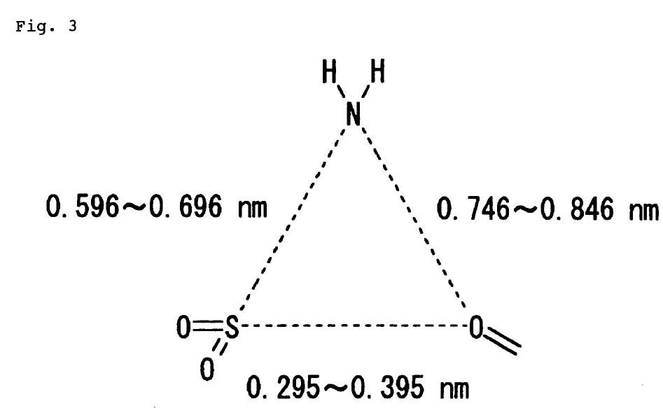

Then, for example, sulfonyl, carbonyl and amino groups of MQPA common to these compounds were specified. For the search of compounds by three-dimensional data base retrieval, available chemical directory (MDL Information Systems, Inc.) was retrieved flexibly by using ISIS-3D (MDL Information Systems, Inc.), making the functional groups and their three-dimensional positions as retrieving conditions according to the three-dimensional retrieving query shown in FIG. 3. The narrowing-down of compounds by ISIS-3D was performed by setting cutoff of the van der Waals energy value on 20.92 KJ/mol. The 119 hit compounds obtained were superposed on the presumed active conformations of thrombin inhibitors; NAPAP, 4-TAPAP and MQPA used for the construction of virtual receptor model, using sulfur atom of sulfonyl, oxygen atom of carbonyl and nitrogen atom of amino group. Then, salt, solvent, etc. contained in compounds were removed, thereby performing the initial docking.

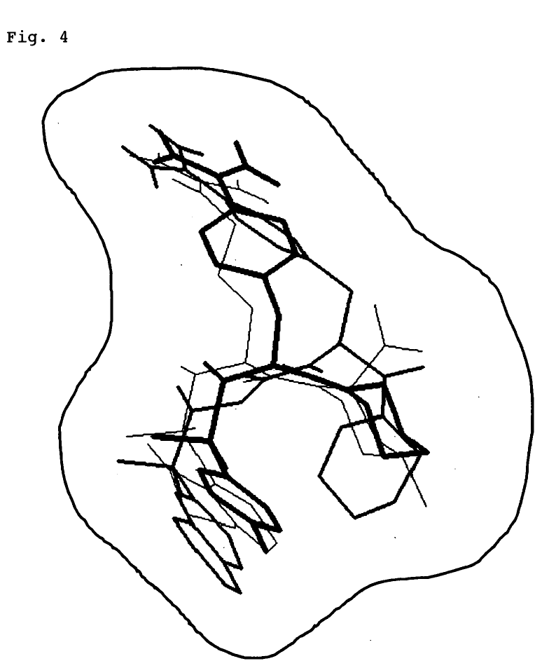

Thereafter, the optimization of the structure of each hit compound was performed under the constrained conditions of virtual receptor model (FIG. 4) to determine the optimum docking, and the value of interaction energy/value of strain energy between virtual receptor model and compound were evaluated. The criterion of this evaluation was made so as the value of interaction energy to be negative and value of strain energy to be 62.76 KJ/mol or less, thereby narrowing down finally to 25 compounds described in Table 2. Therein, 1 compound of trypsin substrate and 2 compounds of trypsin inhibitor being similar protein were contained in addition to 2 compounds of thrombin substrate and 3 compounds of thrombin inhibitor of hit compounds (thrombin activity is known) described in Table 3. The results of example are shown in Tables 2 and 3.

TABLE 2
Compounds selected by three-dimensional retrieval
Strain energy ≦ 62.76 KJ/mol, Interaction energy < 0 KJ/mol

| | Molecular | Known |
|---|---|---|
| Structural formula | formula | bioactivity |
| 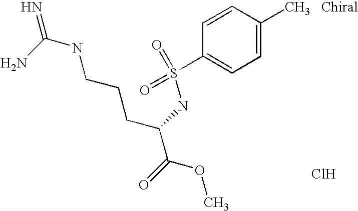 | C14H22N4O4S.HCl | Thrombin substrate |
| 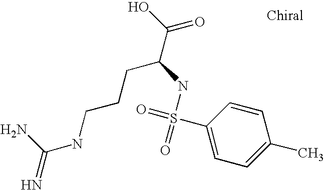 | C13H20N4O4S | |
| 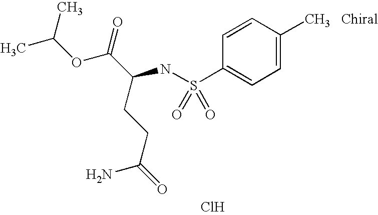 | C15H22N2O5S | |
| 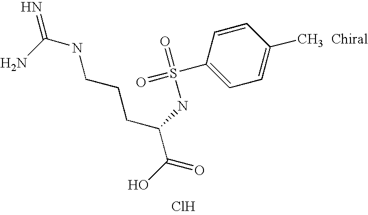 | C13H20N4O4S.HCl | |
| 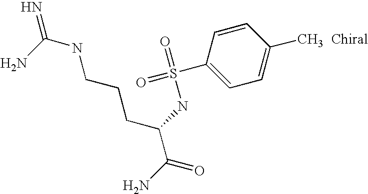 | C13H21N5O3S.HCl | |
| 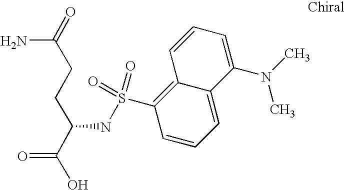 | C17H21N3O5S | |
| 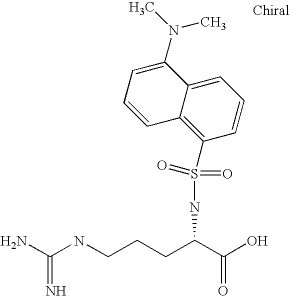 | C18H25N5O4S | |
| 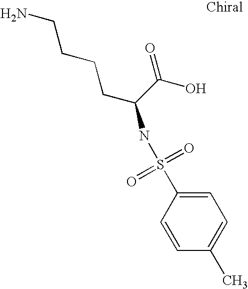 | C13H20N2O4S | |
| 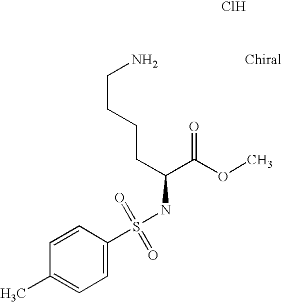 | C14H22N2O4S.HCl | Thrombin substrate |
| 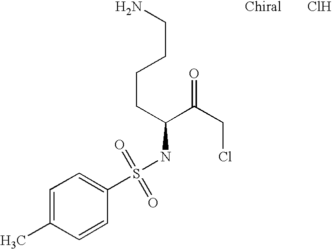 | C14H21ClN2O3S.HCl | Trypsin inhibitor |
| 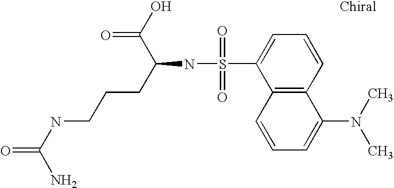 | C18H24N4O5S | |
| 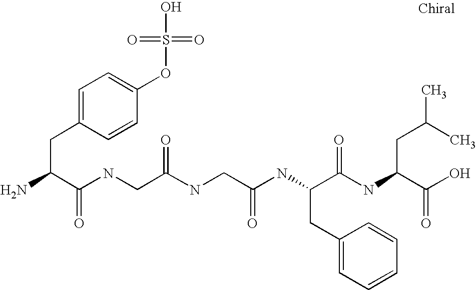 | C28H37N5O10S | Enkephalin leucine |
| 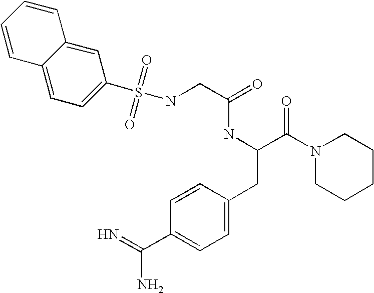 | C27H31N5O4S | Thrombin inhibitor |
| 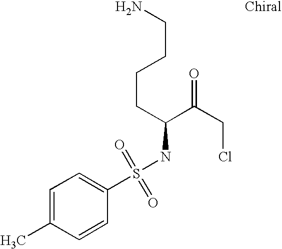 | C14H21ClN2O3S | Trypsin inhibitor |
| 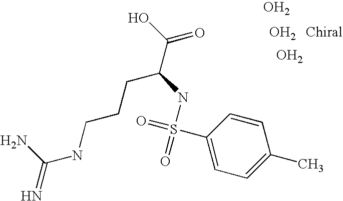 | C13H20N4O4S.3H2O | |
| 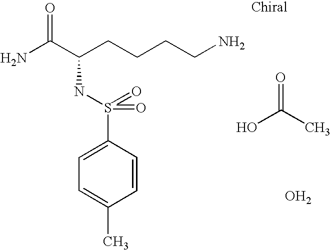 | C13H21N3O3S.C2H4O2.H2O | |
| 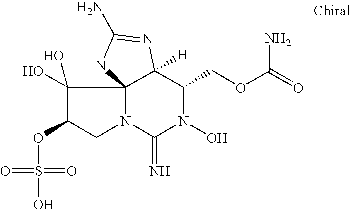 | C10H17N7O9S | Na + Channel probe |
| 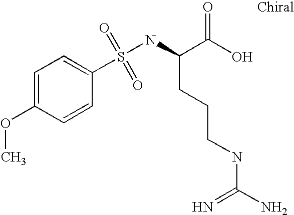 | C13H20N4O5S | |
| 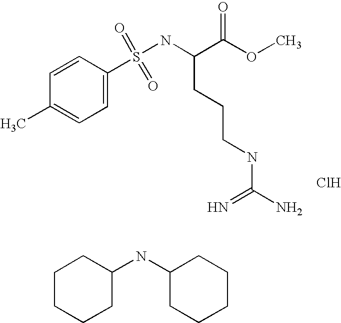 | C14H22N4O4S.HCl | |
| 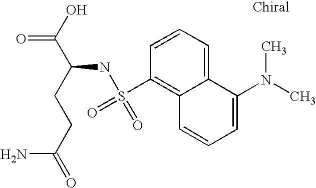 | C17H21N3O5S.C12H23N | |
| 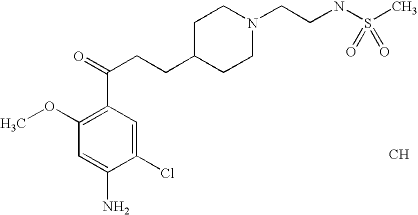 | C18H28ClN3O4S.HCl | 5-HT4 Partial agonist |
| 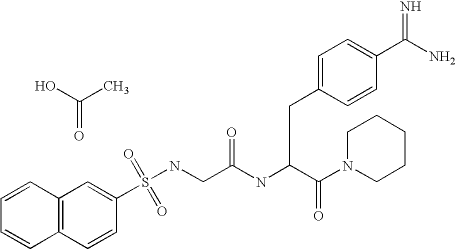 | C27H31N5O4S.C2H4O2 | Thrombin inhibitor |
| 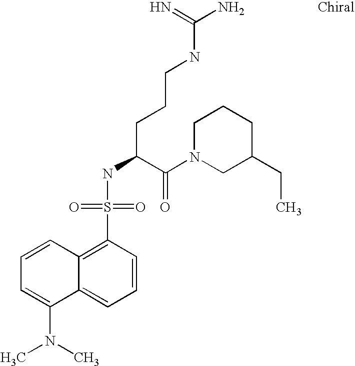 | C25H38N6O3S | Thrombin inhibitor |
| 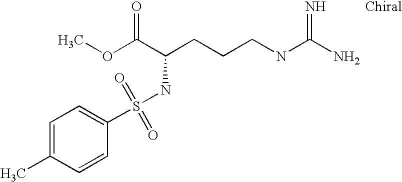 | C14H22N4O4S | Trypsin substrate |
| 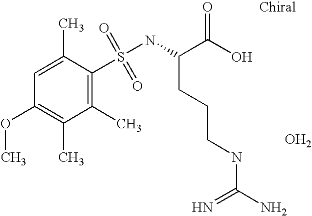 | C16H26N4O5S.H2O | |

TABLE 3

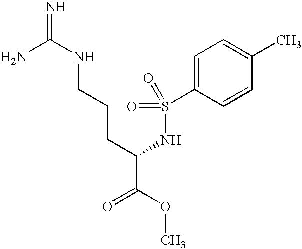

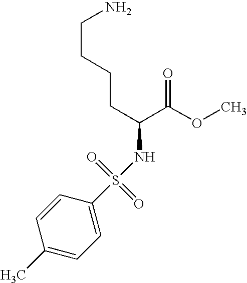

Thrombin substrate

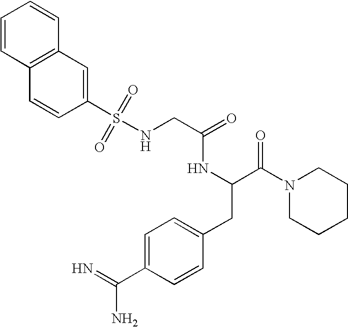

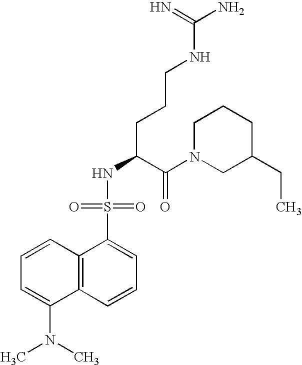

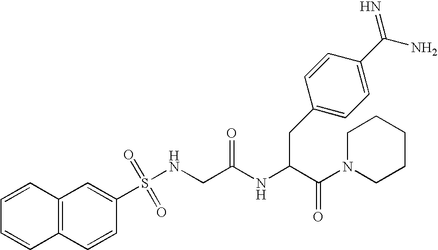

Thrombin inhibitor

Utilizability in the Industry

In the novel retrieving method concerned in the invention, effective selection of novel lead compounds becomes possible by extracting pharmacophores from the superposition between conformers of known compounds, constructing virtual receptor model, and subjecting to docking compounds having hit by three-dimensional data base retrieval based on pharmacophores with the virtual receptor model, which is evaluated with the value of interaction energy/value of strain energy.

### BRIEF DESCRIPTION OF THE DRAWINGS

(FIG. 1) Illustration diagram showing the process of the novel retrieval of lead compounds according to the invention.

(FIG. 2) Illustration diagram showing the superposition of inhibitors of example.

(FIG. 3) Illustration diagram showing the three-dimensional retrieving query of example.

(FIG. 4) Illustration diagram showing the virtual receptor model of inhibitors of example.

1. A method for retrieving lead compounds characterized in that, in the retrieval of compounds with potential to become leads in the compounds of medicinal drugs, agricultural chemicals, etc., the superposition is performed between conformers of compounds and pharmacophores are extracted, the common functional groups and their positions are specified and compounds are searched by three-dimensional data base retrieval, and hit compounds are subjected to docking with the virtual receptor model, which is evaluated with the value of interaction energy and the value of strain energy.

2. A retrieving method characterized in that, in the retrieval of compounds with potential to become leads in the compounds of medicinal drugs, agricultural chemicals, etc.,
(1) conformers of known compounds are sampled,
(2) superposition is performed between conformers of known compounds and virtual receptor model are made out from superposed conformers of known compounds,
(3) pharmacophores are extracted from superposed conformers of known compounds,
(4) three-dimensional retrieving conditions are made out based on the extracted pharmacophores to perform the three-dimensional retrieval,
(5) conformers of compounds having hit by three-dimensional retrieval are superposed making superposed conformers of known compounds as targets, based on the three-dimensional retrieving conditions, and the coordinate is converted,
(6) conformers of hit compounds with coordinate converted are inserted into the virtual receptor model constructed in (2) described above and initial docking is performed, and
(7) optimum docking is determined from the optimization of the structure of hit compounds subjected to initial docking in (6) described above under the constrained conditions of virtual receptor model, and the value of interaction energy/value of strain energy between virtual receptor model and compounds are evaluated, thereby finding out compounds with potential to become leads.
